(19) United States
(12) Patent Application Publication
(10) Pub. No.: US 2008/0255609 A1
(43) Pub. Date: Oct. 16, 2008

(54) LABELED VASCULAR PATCH

(76) Inventor: John C. Opie, Scottsdale, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/735,812

(22) Filed: Apr. 16, 2007

(51) Int. Cl. A61B 17/03

(52) U.S. Cl. 606/213

(57) ABSTRACT

The present invention relates generally to a labeled vascular patch to be applied during surgical repair of blood vessels, which have become diseased by atherosclerosis or injured by trauma. More particularly, the present patch comprises a label, useful for locating the patch during post-operative recovery to assess the success of the surgery and/or during subsequent operations on the same portion of vasculature. The label can comprise any material useful for interacting with imaging energy including, but not limited to Radiation Energy (i.e. X-Rays) electromagnetic energy, sound energy, and light energy, and/or any other energy used for therapeutic imaging.

### FIELD OF INVENTION

The present invention relates generally to a labeled vascular patch to be applied during surgical repair of blood vessels, which have become diseased by atherosclerosis or injured by trauma. More particularly, the present patch comprises a label, useful for locating the patch during post-operative recovery to assess the success of the surgery and/or during subsequent operations on the same portion of vasculature.

### BACKGROUND OF THE INVENTION

Vascular patches are widely used for partial circumferential surgical repair or reconstruction of blood vessels, including both arteries and veins. Common vascular synthetic patch materials include both expanded polytetrafluorethylene (PTFE) and polyethylene-terephthalate (Dacron®). However, PTFE and Dacron® and other vascular patch material have a propensity to thrombose, accelerating atherosclerosis (AS) and patch plaque build up, leading to restenosis or occlusion of the patched blood vessel segment.

Moreover, numerous vascular patches are now produced. These patches have exhibited limited success in reducing this possible restenosis or occlusion. Examples of the numerous vascular patches being produced with various geometries and configurations include typical planar vascular patches, three-part tubular vascular patches with self-tightening collars, tubular vascular patches having a microporous structures allowing partial natural tissue in-growth and limited endothelization, vascular patches with therapeutic transducers to reduce endothelialization, vascular patches with multiple layers, which can be wrapped about one another, flanged vascular patches, tubular vascular patches capable of bridging a vein to an artery, and vascular patches with interlocking support structures.

Due to the above describe problems associated with restenosis or occlusion of patched blood vessels; physicians are often required to reinvestigate the patch area. This reinvestigation is complicated by the fact that it is difficult to know exactly where the patch is located using standard therapeutic imaging devices, like fluoroscopy. Typically, in the past, a physician fixed a metal marker or a metal clip to the skin at the top and bottom of a surgical incision. These markers or clips would provide a range for the physician to look for the patch upon reinvestigation, but such markers or clips may be inaccurate and/or shift over time leading to incorrect interpretation of the patch location.

Thus, a labeled vascular patch, wherein the label assists the physician in distinguishing the patched portion of vasculature from the background tissue is needed and is provided herein.

### SUMMARY OF THE INVENTION

Atherosclerosis (AS) can result in clinical symptomatic peripheral vascular disease (PVD) producing impaired circulation. While some of these disease processes can be managed with remote endovascular percutaneous transluminal angioplasty and stent insertion, in many situations direct access, vascular endarterectomy with patch reconstruction is the preferred procedure. In many situations, as often the case with patients suffering from Chronic Venous Insufficiency (CVI), post implant venography is necessary and precise identification of the original patch implant site is required to ensure that inadvertent balloon venoplasty and/or stenting procedures do not damage the patch implant.

Accordingly, a labeled vascular patch to be applied during surgical repair of blood vessels, wherein the patch comprises a label, useful for locating the patch during post-operative recovery to assess the success of the surgery and/or during subsequent operations on the same portion of vasculature, is disclosed. The label can comprise any material useful for interacting with radiation energy (i.e. X-Rays), sound energy, light energy, electromagnetic energy, and/or any other energy used for therapeutic imaging.

Additionally, in accordance with the various embodiments of the present invention, the label material can be incorporated inside the patch and/or coupled to the surface of the patch in any conceivable pattern. By way of non-limiting example, the label material can provide imagible information regarding the patch (e.g. what the patch is made of), the patient (e.g. age of patient,) and/or the prior surgical procedures (e.g. when and/or how the patch was surgically implanted).

### BRIEF DESCRIPTION OF THE DRAWINGS

A more complete understanding of the present invention, however, may best be obtained by referring to the detailed description when considered in connection with the drawing figures, wherein like numerals denote like elements and wherein:

FIG. 1 illustrates a labeled vascular patch with label material incorporated in said patch in a rectangular fashion in accordance with one exemplary embodiment of the present invention;

FIG. 2 illustrates a labeled vascular patch with label material incorporated in said patch to create horizontal lines in accordance with an exemplary embodiment of the present invention;

FIG. 3 illustrates a labeled vascular patch with label material incorporated in said patch to create vertical lines in accordance with an exemplary embodiment of the present invention;

FIG. 4 illustrates a labeled vascular patch with label material incorporated in said patch to create diagonal lines in accordance with an exemplary embodiment of the present invention;

FIG. 5 illustrates a bullet nosed vascular patch, preferred for arterial implant, with label material incorporated in said patch in accordance with an exemplary embodiment of the present invention;

FIG. 6 illustrates a labeled vascular patch with label material incorporated in said patch to create dots in accordance with an exemplary embodiment of the present invention;

### DETAILED DESCRIPTION OF EXEMPLARY EMBODIMENTS

The detailed description of exemplary embodiments of the invention herein, shows various exemplary embodiments and the best modes, known to the inventors at this time. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those of reasonable skill in the art. Additionally, all included figures are non-limiting illustrations of the exemplary embodiments and modes, which similarly avail themselves to any equivalent modes or embodiments that are known or obvious to those of reasonable skill in the art.

As such, the present invention relates generally to a labeled vascular patch for locating the patch at a post-operative time. For example, the ability to locate the patch and/or obtain various information from the patch to assess the success of the surgery and/or during subsequent operations on the same portion of vasculature is provided by the present invention. Generally, the patch comprises a tubular or a planar layer and a label material. In one exemplary embodiment, the patch can further comprise a biocompatible coating.

More specifically, in accordance with an embodiment of the present invention, the labeled vascular patch comprises: (i) a tubular or a substantially planar layer comprising a first surface for overlying on a blood vessel and a second surface facing away from said blood vessel; and (ii) a label material coupled to and/or incorporated within said tubular or substantially planar layer, wherein the label material interacts with imaging energy thereby allowing the patch to be imaged by a therapeutic imaging device. The label can comprise any material useful for interacting with imaging energy including, but not limited to radiation energy (i.e. X-Rays) electromagnetic energy, sound energy, and light energy, and/or any other energy used for therapeutic imaging.

### Patch Material

With the advent of polytetrafluoroethylene polymer (PTFE) coating, biocompatibility of implantable devices has greatly increased. As such, it is possible to produce vascular patches with polytetrafluoroethylene (PTFE) polymers (i.e. expanded PTFE, most popularly sold under the trade name TEFLON® or GORE-TEX®, and expanded polytetrafluorethylene (PTFE)). Accordingly, the vascular patch recited herein may comprise any biocompatible material, including but not limited to polytetrafluoroethylene (PTFE), and polyethylene-terephthalate (Dacron®).

While these patches typically comprise PTFE or Dacron®, one of reasonable skill in the art will understand that the patch, when implanted without a coating, can be constructed of any biocompatible material and, when coated prior to implant, as discussed below, can be constructed out of any material conducive to conforming to and with an ability to patch a blood vessel.

### Coating Material

As mentioned, an exemplary embodiment of the presently recited vascular patch may further comprise a biocompatible coating, wherein in the coating covers at least a portion of the label material and/or second surface. Moreover, the coating can be a biocompatible polymer. The biocompatible polymer may include, but is not limited to, at least one of a polyurethane, a silicone, a polyurethane-urea, and a silicone-polyurethane polymer.

Currently, many biocompatible coatings are commercially available, but due to complex polymeric nature, these compounds are often referred to by their shorthand trade names, including, but not limited to Pellethane®, a Chronoflex®, a Hydrothane®, an Estane®, an Elast-Eon®, a Texin®, a Biomer®, a Surethane®, a Corethane®, a Carbothane®, a Technoflex®, a Tecothanem®, and a Biospan®.

In general regard to the label coupled to and/or incorporated within the vascular patch, this label material is useful for locating the patch during post-operative recovery to assess the success of the surgery and/or during subsequent operations on the same portion of vasculature. The methodology for coupling and/or incorporating the label material can be completed by any known or as yet unknown means. For example, numerous conventional sewing, knitting, and/or gluing methods may be employed for coupling the label material to and/or incorporating the label material inside the vascular patch.

### Label Material

In accordance with various aspects of the present invention, said label material can be any material useful for interacting with imaging energy including, but not limited to radiation energy (i.e. X-Rays), electromagnetic energy, sound energy, light energy, and/or any other energy used for therapeutic imaging. The mechanism by which the label material interacts with the imaging energy is dependent upon the specific material and energy, as will be individually explained below. Additionally, the label material can be suitable metal alloys, suitable piezoelectric materials, and suitable radio-opaque materials. Optionally, in accordance with an exemplary embodiment, the label material may be biocompatible when coupled to and/or incorporated in the patch without an additional external coating.

Non-limiting examples of piezoelectric materials include crystals, including but not limited to at least one of a tourmaline crystal, a quartz crystal, a topaz crystal, a cane sugar crystal, a Rochelle salt crystal; quartz analogue crystals, including but not limited to berlinite (AlPO₄), gallium orthophosphate (GaPO₄), ceramics with perovskite or tungsten-bronze structures (BaTiO₃, SrTiO₃, Pb(ZrTi)O₃, KNbO₃, LiNbO₃, LiTaO₃, BiFeO₃, NaₓWO₃, Ba₂NaNb₅O₅, Pb₂KNb₅O₁₅); and certain polymers, including but not limited to polyvinylidene fluoride, PVDF.

Non-limiting examples of radio-opaque, metal alloy materials include barium, iodine, bralium, bismuth, and/or tungsten.

### Label Configuration

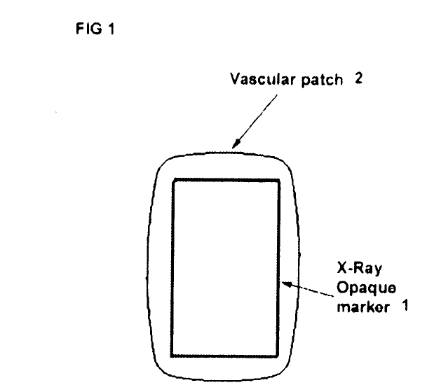

In accordance with various aspects of the present invention, the above described label materials can be coupled to and/or incorporated within the tubular or substantially planar layer of the patch in any orientation, configuration, and/or geometry. For example, referring first to FIG. 1, in accordance with an exemplary embodiment of the present invention, any of the above described label materials can be coupled to and/or incorporated within the tubular or substantially planar layer of the patch in a rectangular pattern 1, as illustrated. In the tubular embodiment, the patch 2 is rolled in to a tube. One of reasonable skill in the art understands that label material 1 may be used to create numerous geometric shapes including, but not limited to squares, circles, ellipses, stars, polygonal shapes, and/or any irregular drawing/shape.

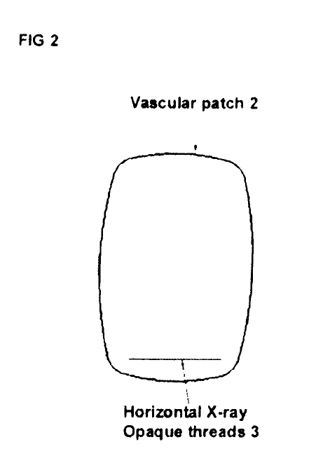

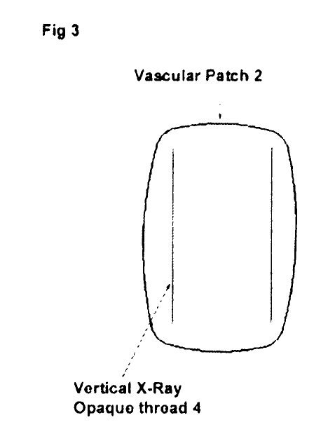

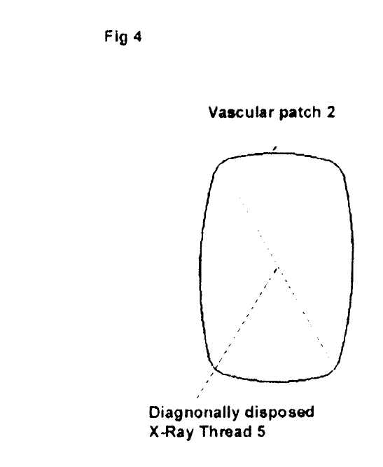

In an exemplary embodiment with reference to FIG. 2, FIG. 3, and FIG. 4, the above described label materials can be coupled to and/or incorporated within the tubular or substantially planar layer of the patch to form a line, 3, 4, and 5, respectively, or group of lines, as illustrated. As illustrated in FIG. 2, the lines can run horizontally across the patch 3, as illustrated in FIG. 3, vertically along the length of the patch 4, or, as illustrated in FIG. 4, the lines can run diagonally from one corner of the patch to a transposed corner across the patch 5. One of reasonable skill in the art understands that label material may be used to create a plurality of lines and that it is possible to form a barcode to store information about the patch (e.g. what the patch is made of), the patient (e.g. age of patient,) and/or the prior surgical procedures (e.g. when and/or how the patch was surgically implanted). In the tubular embodiment, the patch 2 is rolled in to a tube.

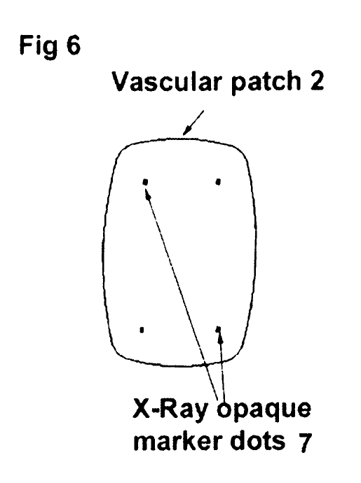

Turning to FIG. 6, in accordance with an exemplary embodiment of the present invention, the above described label materials can be coupled to and/or incorporated within the tubular or substantially planar layer of the patch to form a dot 7, or group of dots, as illustrated. As illustrated in FIG. 6, the dots can be placed anywhere on the patch, preferably at the edges to indicate where the patch starts and ends. One of reasonable skill in the art understands that label material may be used to create a plurality of dots, of any size, and that it is possible to form a picture. Moreover, this picture can store information about the patch (e.g. what the patch is made of), the patient (e.g. age of patient,) and/or the prior surgical procedures (e.g. when and/or how the patch was surgically implanted). In the tubular embodiment, the patch 2 is rolled in to a tube.

One of reasonable skill in the art will understand that the above described label materials can be placed in numerous orientations and geometries on the vascular patch and that any and all of these various placements are contemplated herein.

### Patch Configuration

As mentioned above, numerous patch geometries and configurations are contemplated within the scope of this disclosure including, but not limited to typical planar vascular patches, three-part tubular vascular patches with self-tightening collars, tubular vascular patches having a micro porous structures allowing partial natural tissue in-growth and limited endothelization, vascular patches with therapeutic transducers to reduce endothelialization, vascular patches with multiple layers, which can be wrapped about one another, flanged vascular patches, tubular vascular patches capable of bridging a vein to an artery, and vascular patches with interlocking support structures.

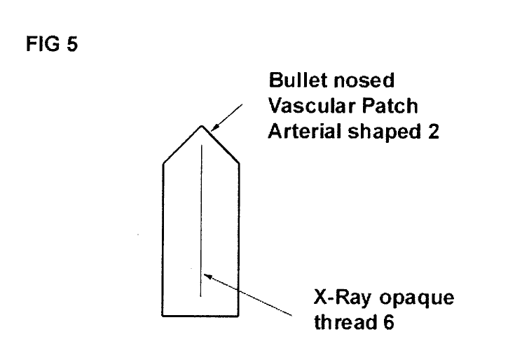

Turning to FIG. 5, in accordance with an exemplary embodiment of the present invention, the above described label materials can be coupled to and/or incorporated within a bullet nosed vascular patch, preferred for arterial implant.

### Imaging Energy and Devices

As mentioned above, in accordance with an exemplary embodiment of the present invention, the energy used to image the label material coupled to and/or incorporated inside the patch can be any imaging energy used for therapeutic imaging including, but not limited to radiation energy (i.e. X-Rays), electromagnetic energy, sound energy, and/or light energy. One of reasonable skill in the art will appreciate that there are numerous methods for therapeutic imaging, which utilize varying energies and energy waves to form images of patient tissue and implanted devices. As such, said person of reasonable skill will appreciate that such imaging means and energies are contemplated within the scope of this disclosure.

For example, most commonly, radiation energy can be employed to create X-Rays, which, in turn, allow for imaging of a patient tissue via diagnostic radiography and/or crystallography. Typically, therapeutic X-Rays imaging uses photographic plates and/or a fluoroscope with a wavelength in the range of 0.01 to 10 nanometers, corresponding to frequencies in the range 30 to 30,000 PHz (10¹⁵ hertz). The radiation energy is able to penetrate patient tissue, but is not able penetrate certain radio-opaque materials, thus these radio-opaque materials interact with the radiation energy by blocking and absorbing the radiation wave energy. This interaction, blocking and absorption, allows the imaging technician to distinguish the radio-opaque material from the surrounding tissue.

Accordingly, a vascular patch with a radio-opaque label material, as taught above, is disclosed, wherein the radio-opaque label material interacts with the X-Ray energy such that an image of at least a portion of the patch can be formed.

In an alternative embodiment, sound energy can be employed to create ultrasonic waves, which, in turn, allow for imaging of a patient tissue via therapeutic ultrasonography (sometimes referred to as medical sonography, more commonly called, “ultrasound”). Ultrasound is a method for imaging used to visualize muscles, tendons, and many internal organs, their size, structure and any pathological lesions. They are also used to visualize a fetus during routine and emergency prenatal care. Notably, ultrasound is generally dismissed as a “safe test” because it does not use ionizing radiation, which imposes hazards, such as cancer production and chromosome breakage. As such, it is well known in the art that many materials, called “piezoelectric” materials, resonate under ultrasonic sound energy (frequency). Thus, the sound energy is able to penetrate patient tissue and interact with the implanted piezoelectric material causing the piezoelectric material to resonate and scatter the sound wave energy. This interaction, resonating and scattering, allows the imaging technician to distinguish the piezoelectric material from the surrounding tissue and is useful for imaging patient tissue and/or implanted devices.

Accordingly, a vascular patch with a piezoelectric label material, as taught above, is disclosed, wherein the piezoelectric label material interacts with the sound energy such that an image of at least a portion of the patch can be formed.

In another exemplary embodiment, magnetic or electromagnetic energy can be employed to create both passive and dynamic electric fields, which, in turn, allow for imaging of a patient tissue via therapeutic magnetic resonance imaging (MRI or sometimes referred to as magnetic resonance tomography, “MRT”). MRI is a method for imaging tissue by subjecting the tissue to multiple passive magnetic fields, such that the tissue is polarized along a given axis (x, y, or z), subsequently a dynamic electromagnetic field is introduced along an axis to excite the tissue, thereby allowing for detailed images to be acquired. Common magnetic field strengths range from 0.3 to 3 tesla (T), although field strengths as high as 9.4 T or higher are used in research scanners and research instruments for animals or only small test tubes range as high as 20 T. As such, it is well known in the art that different materials interact to differing extents with the MRI electromagnetic field energy. For example, magnetic (i.e. ferrous) metals can be problematic due to the strong interaction with the electromagnetic energy used in MRI. Notwithstanding the foregoing, other label materials are useful for imaging and/or providing an internal reference for the MRI operator. Such materials interact with the electromagnetic energy, both passive and dynamic electric fields, used in MRI to a greater extent than the patient tissue such that either an image of at least a portion of the patch can be formed and/or the label material can provide information about location of the patch along the vasculature, preferably the materials interact to a lesser extent than magnetic (i.e. ferrous) metals under MRI.

Accordingly, a vascular patch is disclosed with any label material, as taught above, useful for interacting with magnetic or electromagnetic energy such that either an image of at least a portion of the patch can be formed and/or the label material can provide information about location of the patch along the vasculature.

In another exemplary embodiment, light energy can be employed to allow for imaging of a patient tissue via light wave refraction and/or absorption. It is well established in the art that various wavelengths of light penetrate human epithelial tissue (skin) at differing depths. In this regard, different wavelengths of light energy can be used to image and sense changes in tissue just below the skin. Examples include the use of an exogenous light source and light energy to sense blood chemistry changes. Typically, light imaging is being researched using light in the infrared range, 750 nanometers to 1 mm, and preferably 830 to 850 nanometers. As such, it is well known in the art that different materials interact differently to light energy. For example, different materials refraction and/or absorption light wave energy at differing levels. Thus, the light energy is able to penetrate patient tissue and interact with the implanted label material coupled to and/or incorporated in the vascular patch causing the label material to refract and/or absorb light wave energy. This interaction, refraction and/or absorption of light wave energy, allows the imaging technician to distinguish the label material from the surrounding tissue and is useful for imaging patient tissue and/or implanted devices.

Thus, as stated above, a vascular patch is disclosed with any label material useful for interacting with light energy (i.e. by increasing or decreasing refraction and/or absorption) such that either an image of at least a portion of the patch can be formed and/or the label material can provide information about location of the patch along the vasculature.

### Therapeutic Compounds

In accordance with another exemplary embodiment of the present invention, the disclosed patch can include supplemental chemical compounds. For example, the patch can comprise at least one of a polymeric release agent, an anti-proliferative agent, an anti-thrombotic agent, an anti-migratory agent, an antineoplastic agent, a fibrin growth factor, an anti-bacterial, an anti-biotic, an anti-mitotic agent, an anesthetic agent, an anti-coagulant, a vascular cell growth promoter, a vascular cell growth inhibitor, a cholesterol-lowering agent, a vasodilating agent, and/or an anti-inflammatory compound. It should be noted that these supplemental chemical compounds can be incorporated in to the patch in the planar or tubular layer material, in the label material, and/or the coating. Numerous therapeutic compounds are known in the art and one of reasonable skill in the art will understand that all of these variations are contemplated within the scope of this disclosure.

Finally, as used herein, the terms “comprise”, “comprises”, “comprising”, “having”, “including”, “includes”, or any variation thereof, are intended to reference a non-exclusive inclusion, such that a process, method, article, composition or apparatus that comprises a list of elements does not include only those elements recited, but can also include other elements not expressly listed and equivalents inherently known or obvious to those of reasonable skill in the art. Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters or other operating requirements without departing from the scope of the instant invention and are intended to be included in this disclosure.

Moreover, unless specifically noted, it is the Applicant's intent that the words and phrases in the specification and the claims be given the commonly accepted generic meaning or an ordinary and accustomed meaning used by those of reasonable skill in the applicable arts. In the instance where these meanings differ, the words and phrases in the specification and the claims should be given the broadest possible, generic meaning. If it is intended to limit or narrow these meanings, specific, descriptive adjectives will be used. Absent the use of these specific adjectives, the words and phrases in the specification and the claims should be given the broadest possible meaning. If any other special meaning is intended for any word or phrase, the specification will clearly state and define the special meaning.

What is claimed is:

1. A labeled vascular patch comprising:
a substantially planar layer comprising a first surface for overlying on a blood vessel and a second surface facing away from the blood vessel; and
a label material coupled to the second surface of said substantially planar layer, wherein the label material interacts with imaging energy thereby allowing the patch to be imaged by a therapeutic imaging device.

2. The patch of claim 1, further comprising a coating of biocompatible material, wherein in the coating covers at least a portion of the label material and the second surface.

3. The patch of claim 1, wherein the label material is radio-opaque.

4. The patch of claim 1, wherein the label material is biocompatible.

5. The patch of claim 1, wherein the label material comprises a metal alloy.

6. The patch of claim 1, wherein the imaging energy comprises at least one of electromagnetic energy, sound energy, and light energy.

7. The patch of claim 6, wherein the electromagnetic energy comprises an X-Ray.

8. The patch of claim 7, wherein the therapeutic imaging device comprises a fluoroscope.

9. The patch of claim 6, wherein the therapeutic imaging device comprises at least one of a X-Ray imaging device, a magnetic resonance imaging device, an ultrasound imaging device, and a light imaging device.

10. The patch of claim 1, wherein the label material when imaged produces a visible label, wherein said visible label is at least one of a line, a geometric shape, and a dot.

11. The patch of claim 10, wherein the visible label is a plurality of lines forming a barcode to store information about the patch.

12. The patch of claim 10, wherein the geometric shape comprises at least one of a squares, circles, ellipses, stars, polygonal shapes, and any irregular drawings.

13. The patch of claim 1, wherein the substantially planar biocompatible layer material comprises at least one of a polytetrafluoroethylene and a polyethylene-terephthalate.

14. The patch of claim 2, wherein the biocompatible coating comprises a polymer.

15. The patch of claim 14, wherein the biocompatible polymer coating comprises at least one of a polyurethane, a silicone, a polyurethane-urea, and a silicone-polyurethane polymer.

16. The patch of claim 1, wherein the planar layer is shaped with a bullet nose.

17. The patch of claim 1, further comprising at least one of a polymeric release agent, an anti-proliferative agent, an anti-thrombotic agent, an anti-migratory agent, an antineoplastic agent, a fibrin growth factor, an anti-bacterial, an anti-biotic, an anti-mitotic agent, an anesthetic agent, an anti-coagulant, a vascular cell growth promoter, a vascular cell growth inhibitor, a cholesterol-lowering agent, a vasodilating agent, and/or an anti-inflammatory compound.

18. The patch of claim 1, wherein said label material is incorporated within said substantially planar layer.

19. A labeled vascular patch comprising:
a tubular layer comprising a first surface for overlying on a blood vessel and a second surface facing away from the blood vessel; and
a label material coupled to the second surface of said tubular layer, wherein the label material interacts with imaging energy thereby allowing the patch to be imaged by a therapeutic imaging device.

20. A labeled vascular patch comprising:
a substantially planar layer comprising a first surface for overlying on a blood vessel and a second surface facing away from the blood vessel; and
an energy absorbing label material coupled to the second surface of said substantially planar layer, wherein the label material can be imaged by a therapeutic imaging device.
